(19) United States
(12) Patent Application Publication
(10) Pub. No.: US 2010/0189807 A1
(43) Pub. Date: Jul. 29, 2010

(54) Use of an ozone / oxygen mixture as a primary anticancer therapy via intraperitoneal insufflation

(76) Inventors: Jochen A. Werner, Marburg (DE); Anja-Alexandra Dunne, Grenzach-Wyhlen (DE); Robert Mandic, Stadtallendorf (DE); Eberhard Weihe, Marburg (DE); Michael Bette, Marburg (DE); Siegfried Schulz, Weimar-Niederwalgern (DE)

Correspondence Address: CLARK & BRODY, 1700 Diagonal Road, Suite 510, Alexandria, VA, 22314, US

(21) Appl. No.: 12/320,305

(22) Filed: Jan. 23, 2009

(51) Int. Cl. A61K 33/00; A61P 35/00

(52) U.S. Cl. 424/613

(57) ABSTRACT

Head and neck squamous cell carcinomas (HNSCC) represent a group of metastasizing tumors with a high mortality rate in man and animals. Since the biomolecule ozone was found to inhibit growth of various carcinoma cells in vitro we here applied the highly aggressive and lethal VX2 carcinoma HNSCC tumor model of the New Zealand White rabbit to test whether ozone exerts anti-tumorous effects in vivo. Therapeutic insufflation of medical ozone/oxygen (O₃/O₂) gas mixture into the peritoneum (O₃/O₂-pneumoperitoneum) at an advanced stage of tumor disease led to a survival rate of 7/14 rabbits. Six of the seven surviving rabbits presented full tumor regression and the absence of local or distant lung metastases. Insufflation of pure oxygen (O₂) resulted in a survival rate of 3/13 animals accompanied by full tumor remission in two of the three surviving animals. Of the fourteen sham-treated animals only one had spontaneous tumor remission and survived. No adverse effects or changes in standard blood parameters were observed after repeated intraperitoneal insufflations of the O₃/O₂ or O₂ gas. Animals with O₃/O₂-induced tumor eradication developed tolerance against re-implantation of the VX2 tumor. This could be reversed by immune suppression with a combination of dexamethasone and cyclosporin A suggesting an anti-tumorous effect of O₃/O₂-mediated activation of the body's own immunosurveillance. Although the exact mechanisms of action are still unclear the present data point to O₃/O₂-pneumoperitoneum as a promising new strategy in anticancer therapy.

This is the first report showing a high anti-tumorous potency of medical ozone/oxygen (O₃/O₂) gas mixture in vivo at an advanced stage of tumor disease mediated by an intact immune system which results in highly superior survival, significantly associated with complete remission of cancer. Our findings are in line with new concepts of cancer immunoediting stimulated by the resurgence of the cancer immunosurveillance hypothesis.

For the first time, an ozone/oxygen mixture is used not only adjuvantly to chemo-therapy or irradiation, but as a primary therapy. The gas mixture is insufflated inter-peritoneally in contrast to intramuscular (i.m.) and intravenous (i.v.) injections applied so far.

### FIELD OF THE INVENTION

The present invention refers to the fields of medicine, cellular biology, immunology, and oncology/cancer therapy.

### BACKGROUND AND STATE OF THE ART

Squamous cell carcinomas of the head and neck region (HNSCC) frequently metastasize and show a high mortality rate in man and animals. Being an accepted animal model for studying the progression and metastatic spread of HNSCC, the highly aggressive and lethal VX2 auricular carcinoma model of the New Zealand White (NZW) rabbit^{1,2} was applied in this study. This tumor model was proven to be highly suitable to investigate new therapeutic approaches, since both the HNSCC and the VX2 carcinoma are similar in growth leading to early regional lymph node and subsequent distant metastatic spread.³⁻⁵

As is true for many cancers HNSCC tumor cells somehow evade the body's immune system. This immune escape can partly be explained by the frequently observed down-regulation or loss of MHC class I determinants^{6,7} and an increase in CD4+CD25+ regulatory T cells (T_{reg}) that are found responsible for depressed anti-tumor immunity.⁸⁻¹⁰ Therefore, being able to activate the immunosurveillance toward HNSCC tumor cells should help in recognition and eradication of this tumor entity. Enhancing the immunosurveillance capacity is an emerging concept in recent cancer immunotherapies¹¹⁻¹³ particularly those focusing on immunomodulators or up-regulators of the immune response.¹⁴ To augment the host's immune response against cancer cells, therapies with recombinant cytokines, dendritic cell immunization and tumor antigen vaccination as well as T cell based immunotherapies are currently under investigation.¹⁵⁻¹⁷

Ozone, recently found to be produced endogenously by granulocytes¹⁸, is a gas with complex influence on free radical biology in man and animals.¹⁹ Most research on the biological effects of ozone has focused on its lung toxicity due to inhalation of ambient ozone.^{20,21} Therefore, airway application has limited in vivo studies of medical ozone in human diseases. Interestingly, ozone exhibited potent protective effects on polymicrobial-induced lethal sepsis without any detectable lung toxicity when applied as an O₃/O₂ gas mixture into the peritoneum.²² Additionally, evidence for antibody-catalyzed ozone formation in bacterial killing²³ suggests immune mediated effects of endogenously produced or exogenously applied ozone. Early in vitro studies described ozone as a radiomimetic gas,²⁴ able to selectively inhibit growth of isolated human alveolar, uterine, breast, and endo-metrial carcinomas.²⁵

The common state of the art is referred, for instance, in the following references:
1. Dunne A A, Mandic R, Ramaswamy A, Plehn S, Schulz S, Lippert B M, Moll R, Werner J A. Lymphogenic metastatic spread of auricular VX2 carcinoma in New Zealand white rabbits. Anticancer Res 2002;22:3273-9.
2. Sapundzhiev N, Dunne A A, Ramaswamy A, Werner J A. The auricular VX2 carcinoma: feasibility of complete tumor resection. Anticancer Res 2005;25:4209-14.
3. Shah S A, Dickson J A. Effect of hyperthermia on the immunocompetence of VX2 tumor-bearing rabbits. Cancer Res 1978;38:3523-31.
4. van Es R J, Dullens H F, van der Bilt A, Koole R, Slootweg P J. Evaluation of the VX2 rabbit auricle carcinoma as a model for head and neck cancer in humans. J Craniomaxillofac Surg 2000;28:300-7.
5. Mandic R, Dunne A A, Eikelkamp N, Ramaswamy A, Schulz S, Teymoor-tash A, Sesterhenn A, Moll R, Werner J A. Expression of MMP-3, MMP-13, TIMP-2 and TIMP-3 in the VX2 carcinoma of the New Zealand white rabbit. Anticancer Res 2002;22:3281-4.
6. Mandic R, Lieder A, Sadowski M, Peldszus R, Werner J A. Comparison of surface HLA class I levels in squamous cell carcinoma cell lines of the head and neck. Anticancer Res 2004;24:973-9.
7. Vora A R, Rodgers S, Parker A J, Start R, Rees R C, Murray A K. An immunohistochemical study of altered immunomodulatory molecule expression in head and neck squamous cell carcinoma. Br J Cancer 1997;76:836-44.
8. Schaefer C, Kim G G, Albers A, Hoermann K, Myers E N, Whiteside T L. Characteristics of CD4+CD25+ regulatory T cells in the peripheral circulation of patients with head and neck cancer. Br J Cancer 2005;92:913-20.
9. Morse M A, Clay T M, Mosca P, Lyerly H K. Immunoregulatory T cells in cancer immunotherapy. Expert Opin Biol Ther 2002;2:827-34.
10. Curiel T J. Tregs and rethinking cancer immunotherapy. J Clin Invest 2007;117:1167-74.
11. Waldmann T A. Immunotherapy: past, present and future. Nat Med 2003;9:269-77.
12. Dunn G P, Bruce A T, Ikeda H, Old L J, Schreiber R D. Cancer immunoediting: from immunosurveillance to tumor escape. Nat Immunol 2002;3:991-8.
13. Swann J B, Smyth M J. Immune surveillance of tumors. J Clin Invest 2007;117:1137-46.
14. Nabel G J. Genetic, cellular and immune approaches to disease therapy: past and future. Nat Med 2004; 10:135-41.
15. Young M R. Trials and tribulations of immunotherapy as a treatment option for patients with squamous cell carcinoma of the head and neck. Cancer Immunol Immunother 2004;53:375-82.
16. Bhardwaj N. Harnessing the immune system to treat cancer. J Clin Invest 2007;117:1130-6.
17. Lin W W, Karin M. A cytokine-mediated link between innate immunity, inflammation, and cancer. J Clin Invest 2007;117:1175-83.
18. Babior B M, Takeuchi C, Ruedi J, Gutierrez A, Wentworth P, Jr. Investigating antibody-catalyzed ozone generation by human neutrophils. Proc Natl Acad Sci USA 2003;100:3031-4.
19. Pryor W A, Houk K N, Foote C S, Fukuto J M, Ignarro L J, Squadrito G L, Davies K J. Free radical biology and medicine: it's a gas, man! Am J Physiol Regul Integr Comp Physiol 2006;291:R491-511.
20. Bhalla D K. Ozone-induced lung inflammation and mucosal barrier disruption: toxicology, mechanisms, and implications. J Toxicol Environ Health B Crit Rev 1999;2:31-86.
21. Menzel D B. Ozone: an overview of its toxicity in man and animals. J Toxicol Environ Health 1984;13:183-204.
22. Schulz S, Rodriguez Z Z, Mutters R, Menendez S, Bette M. Repetitive pneumoperitoneum with ozonized oxygen as a preventive in lethal polymicrobial sepsis in rats. Eur Surg Res 2003;35:26-34.
23.Wentworth P, Jr., McDunn J E, Wentworth A D, Takeuchi C, Nieva J, Jones T, Bautista C, Ruedi J M, Gutierrez A, Janda K D, Babior B M, Eschenmoser A, et al. Evidence for antibody-catalyzed ozone formation in bacterial killing and inflammation. Science 2002;298:2195-9.
24. Brinkman R, Lamberts H B. Ozone as a possible radiomimetic gas. Nature 1958;181:1202-3.
25. Sweet F, Kao M S, Lee S C, Hagar W L, Sweet W E. Ozone selectively inhibits growth of human cancer cells. Science 1980;209:931-3.
26. van Es R J, Franssen O, Dullens H F, Bernsen M R, Bosman F, Hennink W E, Slootweg P J. The VX2 carcinoma in the rabbit auricle as an experimental model for intra-arterial embolization of head and neck squamous cell carcinoma with dextran microspheres. Lab Anim 1999;33:175-84.
27. Canadian Council on Animal Care. CCAC guidelines on: choosing an appropriate endpoint in experiments using animals for research, teaching and testinged. Ottawa, Ontario, Canada: Canadian Council on Animal Care, 1998.
28. DCTD., NCI., NIH., DHHS. Common Terminology Criteria for Adverse Events v3.0 (CTCAE): Cancer Therapy Evaluation Program, 2006.
29. Kim W S, Im J G, Chung E C, Han M H, Han J K, Han M C, Ham E K. Hemato-geneous pulmonary metastasis. An experimental study using VX-2 carcinoma in rabbits. Acta Radiol 1993;34:581-5.
30. van Es R J, Baselmans A H, Koten J W, Van Dijk J E, Koole R, Den Otter W. Perilesional IL-2 treatment of a VX2 head-and-neck cancer model can induce a systemic anti-tumour activity. Anticancer Res 2000;20:4163-70.
31. Hough A J, Jr., Hubbard W C, Oates J A. VX2 carcinoma, pulmonary metastases, and neutrophilic leukocytosis. Possible animal model of tumor-associated granulocytosis. Am J Pathol 1983;112:231-7.
32. Cui Z, Willingham M C, Hicks A M, Alexander-Miller M A, Howard T D, Hawkins G A, Miller M S, Weir H M, Du W, DeLong C J. Spontaneous regression of advanced cancer: identification of a unique genetically determined, age-dependent trait in mice. Proc Natl Acad Sci USA 2003;100:6682-7.
33. Hicks A M, Riedlinger G, Willingham M C, Alexander-Miller M A, Von Kap-Herr C, Pettenati M J, Sanders A M, Weir H M, Du W, Kim J, Simpson A J, Old L J, et al. Transferable anticancer innate immunity in spontaneous regression/complete resistance mice. Proc Natl Acad Sci USA 2006;103:7753-8.
34. Hicks A M, Willingham M C, Du W, Pang C S, Old L J, Cui Z. Effector mechanisms of the anti-cancer immune responses of macrophages in SR/CR mice. Cancer Immun 2006;6:11.
35. Pryor W A, Das B, Church D F. The ozonation of unsaturated fatty acids: aldehydes and hydrogen peroxide as products and possible mediators of ozone toxicity. Chem Res Toxicol 1991;4:341-8.
36. Pryor W A, Uppu R M. A kinetic model for the competitive reactions of ozone with amino acid residues in proteins in reverse micelles. J Biol Chem 1993;268:3120-6.
37. Pryor W A. How far does ozone penetrate into the pulmonary air/tissue boundary before it reacts? Free Radic Biol Med 1992;12:83-8.
38. Pryor W A, Squadrito G L, Friedman M. The cascade mechanism to explain ozone toxicity: the role of lipid ozonation products. Free Radic Biol Med 1995;19:935-41.
39. Leikauf G D, Zhao Q, Zhou S, Santrock J. Ozonolysis products of membrane fatty acids activate eicosanoid metabolism in human airway epithelial cells. Am J Respir Cell Mol Biol 1993;9:594-602.
40. Schneider M R, Tang D G, Schirner M, Honn K V. Prostacyclin and its analogues: antimetastatic effects and mechanisms of action. Cancer Metastasis Rev 1994;13:349-64.
41. Hirsch F R, Lippman S M. Advances in the biology of lung cancer chemo-prevention. J Clin Oncol 2005;23:3186-97.
42. Neudecker J, Sauerland S, Neugebauer E, Bergamaschi R, Bonjer H J, Cuschieri A, Fuchs K H, Jacobi C, Jansen F W, Koivusalo A M, Lacy A, McMahon M J, et al. The European Association for Endoscopic Surgery clinical practice guideline on the pneumoperitoneum for laparoscopic surgery. Surg Endosc 2002;16:1121-43.
43. Last J A, Gohil K, Mathrani V C, Kenyon N J. Systemic responses to inhaled ozone in mice: cachexia and down-regulation of liver xenobiotic metabolizing genes. Toxicol Appl Pharmacol 2005;208:117-26.
44. Suckow M A, Douglas F A. The Laboratory Rabbited. Boca Raton, N.Y.: CRC Press, Inc., 1997.

This led us to hypothesize that intraperitoneal application of a medical O₃/O₂ gas mixture stimulates the body's own anti-tumorous immunosurveillance.

### AIM OF THE INVENTION

It is the aim of the present invention to provide a novel substance for the production of a medicament for the primary therapy of highly metastasizing tumors.

### Solution

This aim is achieved, according to the present invention, by the use of a gaseous medical ozone/oxygen mixture for the production of a medicament for the primary therapy of highly metastasizing tumors.

Surprisingly and in contrast to the state of the art, it could be shown that a gaseous medical ozone/oxygen mixture is suitable for the production of a medicament for the primary therapy of highly metastasizing tumors.

In one preferred embodiment, the gaseous medical ozone/oxygen mixture is used for the production of a medicament for the primary therapy of tumors which are highly metastasizing as well as malignant.

In another preferred embodiment, the gaseous medical ozone/oxygen mixture is used for the production of a medicament for the primary therapy of head and neck squamous cell carcinomas (HNSCC). Among these HNSCC tumors is, for instance, the highly aggressive and lethal VX2 carcinoma.

In another preferred embodiment, the gaseous medical ozone/oxygen mixture is used for the production of a medicament for the primary therapy of malignant melanomas.

Preferably, the peritoneal insufflation treatment is carried out as follows: The abdominal wall of the patient is penetrated by a Varess insufflation cannula. The cannula tip is placed in the abdominal cavity between the parietal and the visceral peritoneum. Subsequently, the cannula is connected with the ozone generator, e.g. the Medozonip (see below), and the insufflation can be started.

We have shown for the first time that intraperitoneal application of a medical O₃/O₂ gas mixture eradicates a highly metastasizing cancer at a high rate without exhibiting major adverse effects. The effectiveness of this new method is supported by the complete remission of the primary tumor along with prevention and/or remission of local and distant metastases, that is significantly higher than the observed spontaneous remission rate in sham-treated animals and in line with published observations in the VX2 tumor cell model of the NZW rabbit.⁴ Previously we reported, that local metastases of the parotid lymph node were present with a probability of 62.5% on day 14 and 100% on day 32 after inoculation of the VX2 tumor cell suspension.¹ Thus, the presence of metastases during the therapeutical O₃/O₂ session is highly likely. Interestingly, Hough et al. found VX2 tumor associated granulocytosis to be an indicator of metastatic spread to the lungs.³¹ Similarly, on day 19 after tumor cell inoculation we observed a mild granulocytosis in all animals possibly pointing to the presence of lung metastases. Our observation that all rabbits with remission of the primary tumor were macroscopically free of metastases, exhibiting no cicatrices in the lung indicates that O₃/O₂ therapy is not only able to prevent but also to clear distant metastases. Therefore, we conclude that O₃/O₂ therapy is effective in eradicating the primary as well as to protect against possible regional and distant metastases.

Development of tolerance against re-implantation of VX2 tumors points to an involvement of the immune system in tumor clearance, particularly the adaptive immune system. This assumption is also supported by the observation that perilesional injection of IL-2 resulted in regression of auricular VX2 tumors in rabbits, although at a lower efficiency (25.0%)³⁰ than found in the O₃/O₂-pneumoperitoneum group (42.9%). The loss of tolerance against re-implantation of VX2 tumors after immune suppression further indicates that immunosurveillance is a key player in the eradication of VX2 tumor cells.

Numerous effector cells from the adaptive and from the innate immune system might be responsible for tumor regression. In an animal model with a mice strain that exhibits spontaneous regression/complete resistance (SR/CR) to multiple transplantable cancer cell lines³², leukocytes infiltrating the tumor site consisted of a mixture of multiple subsets of effector cells composed primarily of macrophages, polymorphnuclear cells (PMN), NK cells, and cytotoxic T lymphocytes.³³ Depletion or transfer of specific leukocyte populations of the adaptive or the innate immune system showed, that primarily the innate immune system is involved in successful tumor regression and complete resistance against re-implantation of the tumors in SR/CR mice.³³ Each leukocyte subpopulation exhibits individual tumor cell killing mechanisms by the secretion of different effector molecules such as performs, granzymes or reactive oxygen species (ROS). In the SR/CR mouse model the release of ROS by macrophages was identified as one major effector mechanism of the anti-cancer immune response.³⁴ Therefore, production of ROS by activated macrophages and/or granulocytes could be a possible mechanism of anti-cancer effects induced by the insufflation of O₃/O₂ gas mixture in our study. In this context, the observed mild leukocytosis including granulocytosis after the therapeutical sessions with O₃/O₂ might represent activated leukocytes as potent anticancer effector cells also in the VX2 model.

A local effect of insufflated free ozone in the peritoneal cavity on auricular VX2 tumor cells seems unlikely because of its high and quick reactivity with membranes and numerous cellular biomolecules such as nucleic acids, proteins and unsaturated fatty acids^{35,36}. Regarding the reactivity of ozone with unsaturated fatty acids that are present in cellular membranes and the chemical half life of ozone, it is of interest that free ozone in the lung is unable to penetrate a biological fluid layer thicker than 0.1 μm.³⁷ Additionally, transport of free ozone via the blood route and thus the generation of ROS by free ozone at the tumor site appears not likely, because any remaining free ozone which did not interact with unsaturated fatty acids or proteins in the serosa and penetrates the peritoneum, will at last completely react with proteins and membranes of local cells of the adventitia attached to the peritoneum. Therefore, lipid ozonation products originated from the reaction of ozone with several biomolecules³⁸ at the serosa are most likely the mediators responsible for triggering the observed anti-tumorigenic effects in the VX2 model. In this context, intraperitoneal insufflated O₃/O₂ gas mixture might react on the arachidonic acid metabolism in the mesothelium consisting of different mesothelial cell types, which is reminiscent to the situation in the eicosanoid metabolism of human airway epithelial cells after exposure to ozone.³⁹ Several components of the prostanoid biosynthesis pathway were found to exhibit oncolytic and anti-metastatic effects on tumor cells and tumor progression.^{40,41} Encouraging studies are currently under way to analyze changes in the plasma levels of different arachidonic acid metabolites during the course of O₃/O₂ insufflation in rabbits (unpublished data).

Since O₃/O₂-pneumoperitoneum is a new therapeutic approach it is of high importance to analyze possible adverse side effects. Acute side effects due to high IAP that might occur during the period of insufflation are unlikely, since the measured peak pressure was below 5 mbar, classified as low IAP in laparoscopic surgery.⁴² The slight and transient reduction in body weight during and shortly after the therapeutic treatment might represent a mild adverse affect (weight loss 5-10%, grade I) comparable to that observed in mice (weight loss up to 14%, grade II) that had inhaled ozone.⁴³ Since no severe side effects such as sustained reduction in body weight and gas embolism, fever, diarrhea or peritonitis were observed, it appears that the O₃/O₂ therapy is relatively safe. Late adverse effects of O₃/O₂-pneumoperitoneum treatment are rather unlikely, since two rabbits from a previous pilot study developed no clinical signs after more than 5 years of tumor remission (not shown).

Taken together, insufflation of medical O₃/O₂ gas mixture into the peritoneal cavity appears to be a highly promising new tool in the treatment of cancer. The exact mechanisms underlying the proposed enhancement of the O₃/O₂-pneumoperitoneum in immunosurveillance are still unknown. Clearly, more work is required to clarify the basal biochemical, physiological and immunological mechanisms underlying the anti-tumorigenic and anti-metastatic effect of the O₃/O₂-pneumoperitoneum on VX2 tumors. Certainly, the effectiveness of this novel therapeutic approach—including possible yet unknown side effects—has to be proven on other tumor animal models of different tumor types before eventually entering clinical trials in humans.

### Embodiments

1. Therapy of VX2 Carcinoma in Rabbits by Intraperitoneal Ozone/Oxygen-Insufflation

Material and Methods

Animals

The study comprises 55 healthy adult Iffa Credo New Zealand White (NZW) out-breed rabbits in a body weight range from 2.0 to 3.0 kg, purchased from Charles River WIGA, Sulzfeld, Germany. All animals were kept in rooms with standardized air conditioning: at 20-22° C., 50%-60% humidity and a 12 h artificial day/night rhythm. Each rabbit was held in an individual steel cage, was fed with 100 g complete diet food pellets per day and had free access to acidified (hydrochloride acid, pH 2.7) tap water ad libitum. Animals could acclimatize for at least 5 days before the experimental procedure was started.

Experimental Design and Tumor Transplantation

The study was in accordance with the guidelines of FELASA and was approved by the RP Giessen (V 54-19 c 20-15(1) MR, Nr. 24/2005), Germany, according to the German Animal Protection Law. The rabbits were randomly divided into three experimental groups: i) animals that received an O₃/O₂ gas mixture therapy (n=14 animals), ii) animals receiving O₂ gas therapy (n=14 animals), iii) sham-treated animals that were treated similar to the other groups but receiving no gas insufflation (n=14 animals). Fourteen additional NZW rabbits were used as donors in which VX2 tumor cells were propagated by intramuscular passages. Fresh tumor cell suspension was derived from the hind leg tumor of donor rabbits as described previously.⁴⁴ The animals of all experimental groups were sedated with 5 mg/kg body weight xylazine intramuscularly (Rompun®, Bayer Vital GmbH, Leverkusen, Germany) and received a slow subcutaneous injection of 1-2×10⁷ vital tumor cells suspended in a volume of 0.3 ml into the area between the central auricular artery and the caudal margin at the dorsal middle-third of the right ear, as described in detail elsewhere.^{1,4,26} Tumor growth was allowed for fourteen days (for experimental design see FIG. 1a). On day 14, the daily therapeutic treatment with O₃/O₂ gas mixture or pure O₂ was initiated lasting for a period of five days. For this purpose, animals were premedicated with 0.1 ml/kg body weight glycopyrrolate (Robinul®, Riemser Arzneimittel A G, Greifswald, Germany) subcutaneously, followed by a short anesthesia by application of 0.3 ml/kg body weight medetomidine hydrochloride (Domitor®, Pfizer GmbH, Karlsruhe, Germany) intramuscularly plus 0.3 to 0.6 ml/kg body weight propofol (Propofol 1% Fresenius, Fresenius Kabi Deutschland GmbH, Germany) intravenously into the ear vein. After treatment, 0.3 ml/kg body weight of the antagonist atipamezole hydrochloride (Antisedan®, Pfizer GmbH, Karlsruhe, Germany) was applied intravenously. The sham group was treated likewise and served as a control. Animals of the O₃/O₂ group received an insufflation of the O₃/O₂ gas mixture and rabbits of the O₂ group received an insufflation of pure medical oxygen into the peritoneum. The ozone gas processor Medozon^{IP} (Herrmann Apparatebau GmbH, Kleinwallstadt, Germany; license holder) was used for the generation and intraperitoneal insufflation of the O₃/O₂ gas mixture and the O₂ gas volume. An integrated intraabdominal pressure control (IAPC) in the Medozon^{IP} gas processor avoids high abdominal pressures during the insufflation process. This novel Medozon^{IP} gas processor has been invented by S. Schulz, the first author of this report and was recently patented (Az: DE102004017599.3; PCT/DR2005/000597; EP05740628.2-2310; patent holder Transmit GmbH Giessen, Germany). The freshly synthesized O₃/O₂ gas mixture or pure medical O₂ gas was immediately insufflated from the Medozon^{IP} O₃/O₂ gas generator into the peritoneum of the rabbits via a sterile Ozon-Kit (Ozone-Set ip REF HAB #18052, Herrmann Apparatebau GmbH, Kleinwallstadt, Germany). The Ozon-Kit consists of a plastic tube of 150 mm length and a sterile filter at the side of which it is connected to the outlet of the Medozon^{IP} generator. The tube of the Ozon-Kit was automatically flushed with approximately 10 ml of the requested gas to remove air from the tube when the stopcock was opened to the “backflush” of the Medozon^{IP} generator. The O₃/O₂ gas mixture was applied in standardized volume of 80 ml/kg body weight containing 50 μg O₃ per ml gas mixture corresponding to a contingent of 2.5% O₃ and 97.5% medical O₂. The stopcock connected to a Vasofix R Braunuele R (17G, Braun Melsungen A G, Melsungen, Germany) that was implanted into the right lower quadrant of the abdomen was used for IAPC during the insufflation process and allowed control of the actual IAP. Analogously, animals of the sham group were anesthetized and connected to the Medozon^{IP} processor, but received no gas insufflation. All animals were observed until day 90 post tumor cell transplantation. Their general health condition was monitored and the body weight, the size of the primary right ear tumor and the parotid lymph nodes were measured daily using the calliper Sylvac SA, Swiss system. To prevent animals from early death due to severe local infection at the tumor site or massive bleeding in the course of primary tumor development, ablation of the ear 1 to 2 cm proximal to the tumor was performed under anesthesia with 0.1 ml/kg body weight glycopyrrolate subcutaneously, followed by intramuscular injection with a combination of 5 mg/kg body weight xylazine and 70 mg/kg body weight ketamine. Signs of distress, pain or cachexy—defined as weight loss above 20%—were always criteria for euthanization as recommended by the Canadian Council on Animal Care²⁷. Furthermore, criteria for grading of adverse effects before and after O₃/O₂ therapy were recorded according to the Cancer Therapy Evaluation Program (CTEP)²⁸ during the whole period of observation.

Immune Suppression

The six rabbits of this report with complete remission of the VX2 tumor after insufflation of O₃/O₂ gas mixture into the peritoneum (O₃/O₂-rem) were divided into two groups. Animals in one group were immune suppressed by a combination of dexamethasone (Dex; Dexa, Jenapharm, Jena, Germany; 1.5 mg/kg body weight; subcutaneous) and cyclosporin A (CSA, Sandimmun®, Novartis Pharma, GmbH, Nuremberg, Germany, 20 mg/kg body weight, subcutaneous) (O₃/O₂-rem+Dex/CSA, n=3), animals of the second group were sham-treated (O₃/O₂-rem+sham, n=3). A one-time dosage of dexamethasone was given on day minus 2 subcutaneously together with the first CSA injection (FIG. 1b). Immune suppression was maintained by daily applications of 20 mg CSA/kg body weight given subcutaneously for 11 consecutive days. One additional rabbit that received the tumor suspension for the first time (control+sham) was used as control for the aggressiveness of the tumor suspension. Another rabbit that was immune suppressed by Dex/CSA (control+Dex/CSA) was used to monitor for possible effects of the immune suppressant agents on the take rate of the tumor. All rabbits used in this experimental part of the study received the same VX2 cell suspension derived from one donor rabbit. Two days after application of the first injection of the immune suppressant agents or sham treatment, all rabbits received an inoculation of VX2 tumor cell suspension similar to the inoculation described above. To enhance the number of possible tumors, VX2 suspension was injected in both ears. The re-take rate of the re-implanted tumor cell suspension was determined on day 14 after inoculation.

Computed Tomography

Rabbits were premedicated with 0.1 ml/kg body weight glycopyrrolate subcutaneously and anaesthetized with a combination of 5 mg/kg xylazine and 30 mg/kg ketamine by intramuscular injection. Computed tomography of the thorax was generated with the Siemens Somatom Plus 4 (Siemens, Erlangen, Germany).

Blood Parameters

Arterial blood samples from the central auricular artery of the left tumor free ear were taken on day 14 post inoculation (directly before the first gas insufflation/sham treatment) and on day 19 post inoculation (24 h after the last gas insufflation/sham treatment). Additional blood samples were taken on day 90 from the O₃/O₂-rem animals (FIG. 1a). For hematological investigations we used an autoanalyzer (Vet abc™ Animal Blood Counter, ABX Diagnostics, Goettingen, Germany), that has been carefully adjusted and validated for the analysis of rabbit blood. For differential blood counts the RBC count was determined together with the hematocrit (HCT) and hemoglobin (HGB). White blood cells were further differentiated into granulocytes, lymphocytes and monocytes. Data were expressed as mean values±SEM. For clinical chemistry investigations creatinine, GOT, and GPT values were measured with the Reflovet® Plus system (Roche Diagnostics GmbH, Mannheim, Germany) to monitor for kidney and liver functions.

Statistics

For comparison of survival rates of the three experimental groups the logrank test was performed considering p values<0.05 as significant. The survival probability of rabbits calculated from the time of tumor cell inoculation until day 90 was depicted according to the Kaplan-Meier method. The time to tumor clearance (TTC) probability was calculated from the time when the size of the solid auricular tumor dropped under 5% of the volume measured on day 14 post tumor cell inoculation, a time point when a solid auricular tumor had developed and gas insufflation therapies or sham treatment started. Statistical differences in mean body weight between the experimental groups were evaluated at daily intervals with the unpaired Student's t test with two tail p values. In order to evaluate possible adverse effects due to the insufflation of O₃/O₂, pure O₂ or sham treatment, blood parameter were measured shortly before the first gas insufflation or sham treatment (day 14) and 24 hrs after the last gas insufflation or sham treatment (day 19). Statistic differences within each experimental group were calculated by the paired Student's t test with two tail P values. The GraphPad Prism version 4.00 for Windows (GraphPad Software, San Diego Calif. USA, www.graphpad.com) was used for all statistical calculations.

Results

To test the hypothesis that intraperitoneal application of a medical O₃/O₂ gas mixture is an effective anti-tumor approach, we subjected 42 NZW rabbits to unilateral inoculation with a VX2 tumor cell suspension into the subcutis of the right ear. All 42 animals developed a solid VX2 tumor at the site of inoculation. One animal died prior to the therapeutic session due to undefined reasons. On day 14 after inoculation, when tumors had reached a mean size of 6082 mm³±515 mm³, rabbits were randomly divided into three groups of 14 or 13 animals each. The group, that received a daily intraperitoneal insufflation of the O₃/O₂ gas mixture over five consecutive days, exhibited a survival rate of 50.0% (FIG. 2a). The probability of survival, calculated by the logrank test, demonstrated a significant increase (p value=0.0006) in the O₃/O₂-treated group compared to the sham-treated group (7.1%) (FIG. 2a). Difference of the survival probability between the O₃/O₂-treated group and the O₂-treated group were calculated as non-significant but the p value of 0.0559 suggest that the insufflation of the O₃/O₂ gas mixture is more efficient than that of pure medical O₂ (FIG. 2a). The mean survival time for O₃/O₂-treated rabbits was 87.5 days, which is close to the end point of the total observation period (day 90). In sharp contrast, the mean survival time for O₂-treated rabbits was 51 days which is comparable to that of the one survivor in the sham group (47.5 days) that exhibited a spontaneous remission of the tumor. FIG. 2b depicts the time course of tumor clearance (TTC) in the three experimental groups. The TTC in the O₃/O₂-treated group (42.9%) is significantly higher (p value=0.0243) than that of the sham-treated group (7.1%). All six O₃/O₂-treated rabbits that had cleared the auricular tumor within two to three weeks after therapeutic intervention were completely free of cancer. The spontaneous remission of the tumor in one of the sham-treated rabbits on day 45 and the tumor clearance observed in the O₂-treated animals on day 68 and 85 occurred at a much later time point than in the O₃/O₂-treated rabbits. This suggests a specific therapeutic effect of the O₃/O₂ gas mixture on tumor clearance in this group, however, there was rather a trend than a significant difference between the TTCs of O₃/O₂-treated and O₂-treated animals (p value=0.0665).

FIG. 3 shows a representative sequence of different tumor stages demonstrating primary tumor regression of the auricular tumor after O₃/O₂ treatment until complete remission (FIG. 3a) or progression of the tumor to the final stage, which is characterized by strong ulceration and massive bleeding shortly before death (FIG. 3b).

To analyze, whether the treatment results in a regression and/or prevention of distant lung metastases in the thorax, which is typical for advanced VX2 tumor disease ease in rabbits,^{4,29} we scanned the complete thorax of the animals with regression of the primary tumor for the presence of metastases using computed tomography (CT). In all three experimental groups lung metastases were absent in rabbits with remission of the primary auricular tumor (FIG. 4a). Contrary to the rabbits with tumor remission, all animals with persisting auricular tumors at day 90 regularly exhibited numerous lung metastases (FIG. 4b).

The observation that re-implanted VX2 tumor cells were rejected in rabbits with tumor remission after perilesional treatment with IL-2³⁰ prompted us to test whether re-implantation of VX2 carcinoma cells will also fail in rabbits with tumor remission after O₃/O₂ treatment. To test this, we divided the six O₃/O₂-rem animals from the present experiment into two groups. Each animal received a bi-auricular injection of the VX2 tumor cell suspension to raise the number of possible tumors. One group was immune suppressed by Dex and CSA (n=3), the other group was sham-treated as described above (n=3). As expected, all sham-treated animals were protected for re-take of the VX2 tumors, since no auricular tumors developed within the observation period of 90 days (Table 1). In sharp contrast, immune suppressed animals developed tumors in 4 out of 6 tumor cell re-inoculations (Table 1). Tumor growth and size in these animals did not show any difference to the auricular tumors previously measured in immune competent rabbits of the sham group.

TABLE 1
Reuptake rate of auricular VX2 tumors.

| | animals | tumors* | mean tumor |
|---|---|---|---|
| experimental group | [n] | [n] | volume [mm³] |
| O₃/O₂-rem + Dex/CSA | 3 | 4/6 | 3089 |
| O₃/O₂-rem + sham | 3 | 0/6 | <200^{#} |
| control + Dex/CSA | 1 | 1/2 | 1466 |
| control + sham | 1 | 2/2 | 5657 |

Bi-auricular re-implantation of VX2 tumor cells in rabbits with complete tumor regression (O₃/O₂-rem) and subsequent treatment with (O₃/O₂-rem+Dex/CSA) or without (O₃/O₂-rem+sham) dexamethasone and cyclosporin A. The take rate of tumors measured in two rabbits served as a baseline control: one rabbit (control+Dex/CSA) received the tumor suspension for the first time and was additionally immune suppressed, whereas the second rabbit (control+sham) was not immune suppressed.

*Due to the bi-auricular transplantation of VX2 cell suspension, two tumors per animal are possible. ^{#}The mean value of less than 200 mm³ represents the basal swelling that occurs after implantation of the VX2 tumor cell suspension. Therefore volumes measuring less than 200 mm³ are considered VX2 tumor negative.

To test whether O₃/O₂ therapy has any adverse effects, we measured the intraabdominal pressure (IAP) during the insufflation process and analyzed weight development and some of the major blood parameters that frequently serve as indicators for adverse effects by the common toxicity criteria.²⁸ Measurement of the IAP in our rabbits revealed no pressure above 5 mbar at any time. Body weight analysis revealed a maximal drop of about 8.1% (p<0.001) in the O₃/O₂-treated group compared to the mean body weight on day 14 directly before starting the first therapeutic session (FIG. 5). In comparison, the mean body weight dropped maximally 5.0% (p<0.072) in the O₂-treated group and 1.5% (p<0.299) in the sham-treated group (FIG. 5). After the end of the daily therapeutic gas-insufflations or sham interventions, the body weight increased in all groups and reached comparable values on day 42.

Measurement of blood parameters on day 19 (24 hrs after the last O₃/O₂ gas insufflation or sham treatment) exhibited a slight increase in WBC count (FIG. 5b). Other values such as RBC count, hemoglobin, hematocrit, GOT and GPT remained within the physiological range (Table 2).

TABLE 2
Hematological and clinical chemistry parameters

| | | | | | | | O₃/O₂ | | |
|---|---|---|---|---|---|---|---|---|---|
| | O₃/O₂ | | O₂ | | Sham | | rem | | |
| | (n = 14) | | (n = 13) | | (n = 14) | | (n = 6) | | |
| parameter | d 14 | d 19 | d 14 | d 19 | d 14 | d 19 | d 90 | reference values (44) | |
| WBC (total) | 8.6 | 11.4*** | 8.5 | 10.6* | 8.6 | 10.7 | 7.6 | 2.5-9.8 | (10³/mm³) |
| granulocytes | 3.4 | 5.5*** | 3.1 | 4.8* | 3.5 | 4.9* | 1.8 | 1.6-3.7 | (10³/mm³) |
| lymphocytes | 4.9 | 5.7 | 5.3 | 5.4 | 4.9 | 5.6* | 5.6 | 3.3-7.0 | (10³/mm³) |
| monocytes | 0.2 | 0.3* | 0.22 | 0.25 | 0.2 | 0.3 | 0.1 | 0.0-0.4 | (10³/mm³) |
| RBC | 5.85 | 5.55 | 5.68 | 5.31 | 5.64 | 5.59 | 5.91 | 5.20-6.80 | (10⁶/mm³) |
| hemoglobin | 11.7 | 11.6 | 12.5 | 11.1 | 10.0 | 11.5 | 12.9 | 9.8-14.0 | (g/dl) |
| HCT | 38.4 | 36.4 | 39.0 | 38.9 | 35.8 | 36.1 | 40.2 | 36.0-47.0 | (%) |
| creatinine | 0.736 | 0.863** | 0.743 | 0.935 | 0.787 | 0.800 | 0.848 | 0.5-2.6 | (mg/dl) |
| GOT | 17.39 | 13.73 | 14.20 | 8.94 | 15.15 | 13.72 | 29.72 | 8.0-56 | (U/l) |
| GPT | 34.7 | 27.3 | 34.7 | 21.6 | 22.9 | 21.0 | 74.9 | 18.0-123.0 | (U/l) |

Effect of repetitive O₃/O₂-pneumoperitoneum on standard laboratory blood parameters.⁴⁴ Arterial blood samples were taken on day 14 post inoculation (directly before the first O₃/O₂ insufflation, O₂ insufflation or sham treatment) and on day 19 post inoculation (24 h after the last gas insufflation or sham treatment). To consider possible long-term effects of O₃/O₂-pneumoperitoneum, blood parameters of all O₃/O₂-rem animals were also measured on day 90 representing the end of the observation period. Statistic differences between day 14 and day 19 in each experimental group were calculated with the paired Student's t test and statistically significant changes were marked with p<0.05*; p<0.01**; p<0.001***. Abbreviations: WBC, white blood cells; RBC, red blood cells, HCT, hematocrit; GOT, glutamic oxaloacetic transaminase; GPT, glutamic pyruvic transaminase.

2. Therapy of a Dog Suffering From a Malignant Melanoma by Intraperitoneal Ozone/Oxygen Insufflation

A dog suffering from a malignant melanoma in the nasal snout region was treated intraperitoneally by ozone/oxygen insufflation anlagously to the treatment of the New Zealand White rabbits suffering from VX2 tumors. The intraperitoneal ozone/oxygen insufflation led to a total regression of the melanoma.

### FIGURE LEGENDS

FIG. 1

Depicted is the experimental design of this study for (a) the treatment scheme and (b) the retake rate after immune suppression of the rabbits. *time points of blood withdrawal

FIG. 2

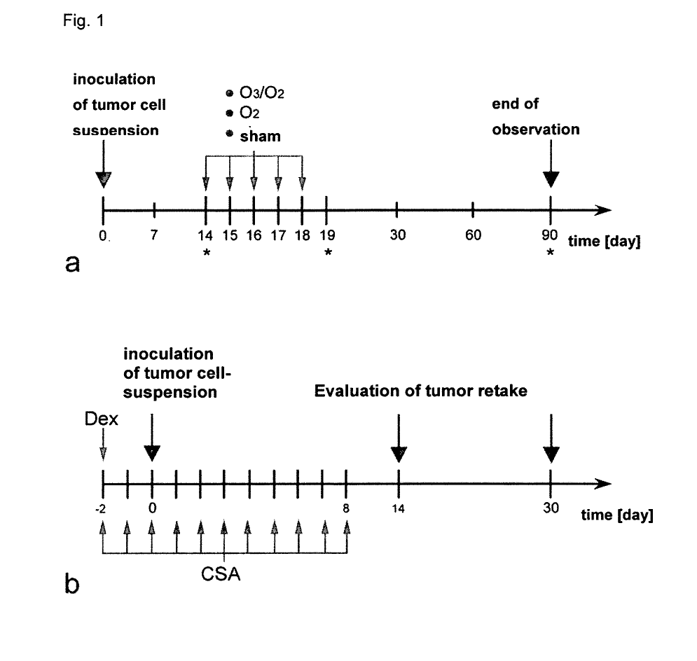

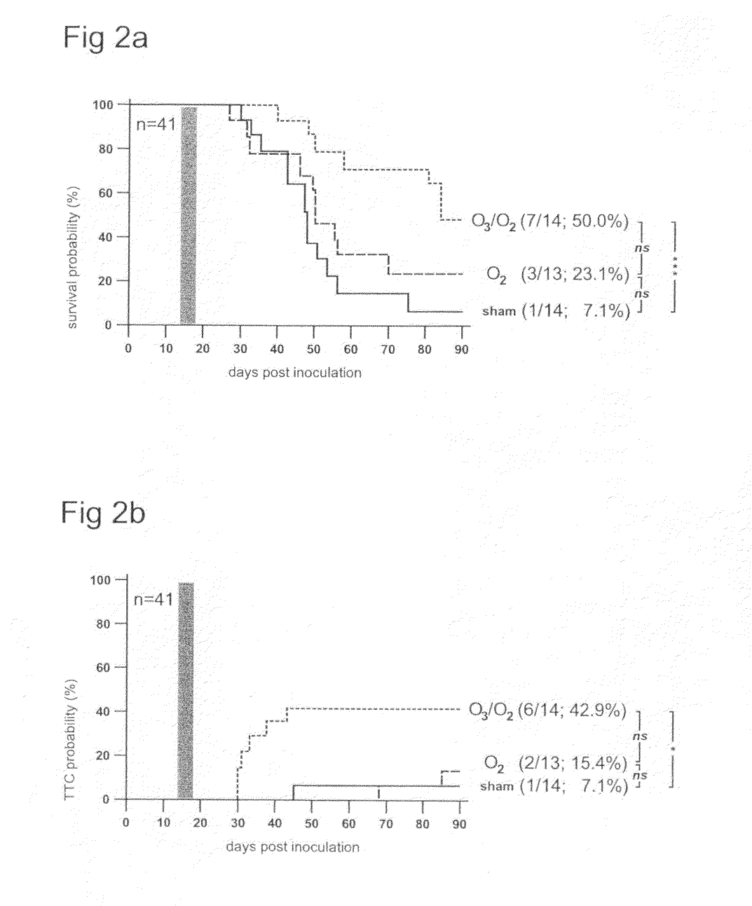

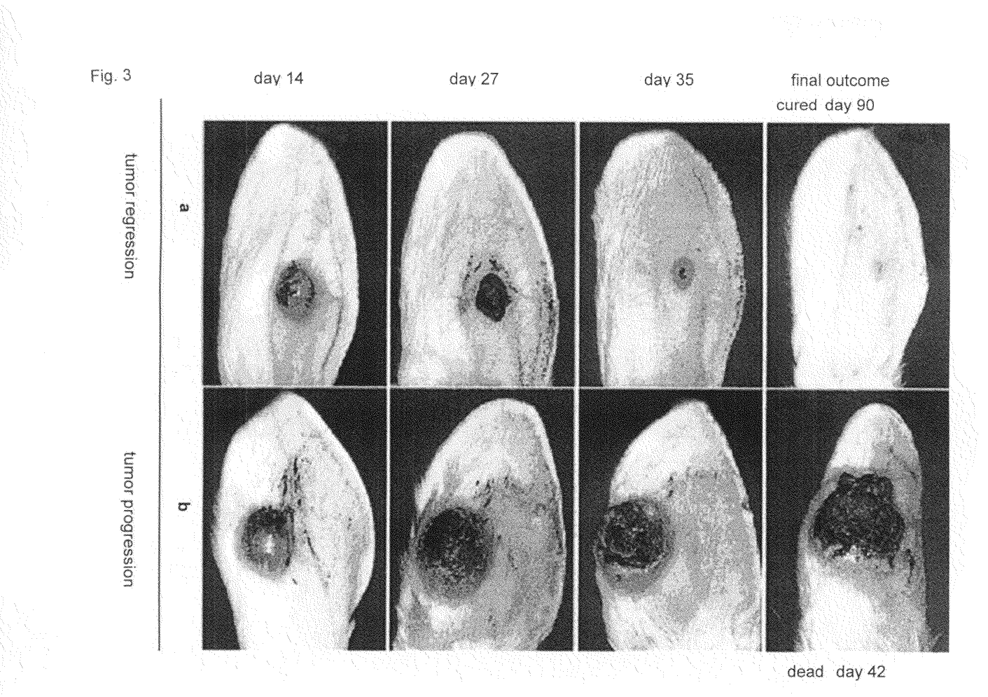

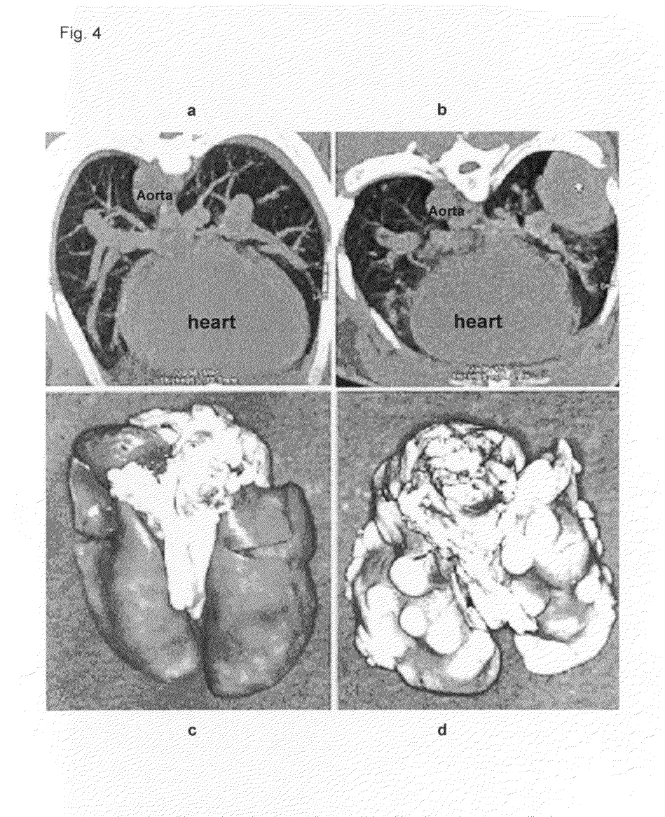

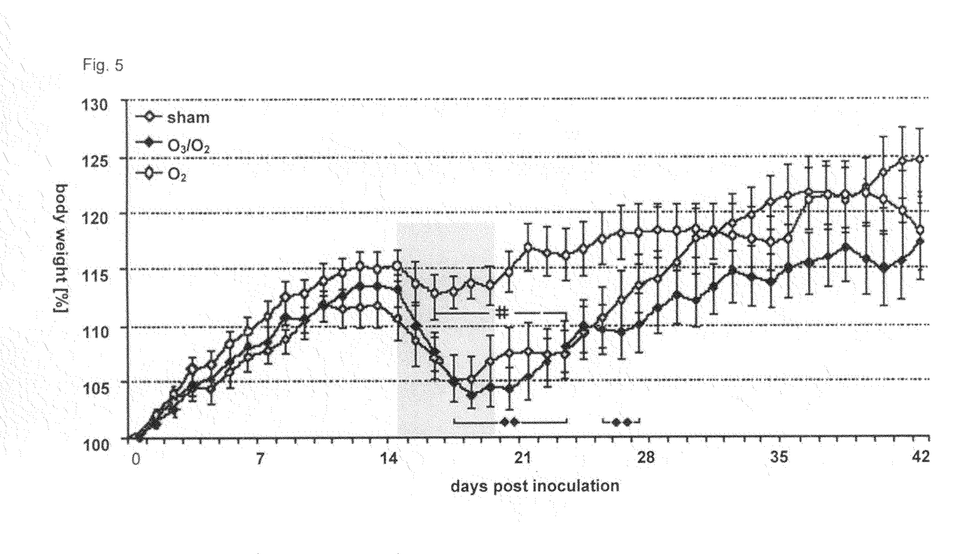

Kaplan-Meier plots showing the survival probability (a) and the time to tumor clearance probability (TTC, (b)) of rabbits (n=41) that have developed a solid auricular VX2 tumor on day 14 after VX2 tumor inoculation. The period of treatment initiation is marked with a gray box. (a) The survival probability of rabbits receiving the O₃/O₂ treatment was superior to sham-treated rabbits (p=0.0006) but was not significantly different from that of the O₂-treated group (p=0.0559) as calculated by the logrank test. The survival probability of O₂-treated rabbits did not differ significantly from that of sham-treated rabbits (p=0.2448). (b) The TTC probability significantly varied between O₃/O₂-treated and sham-treated rabbits (p=0.0243) but not between O₃/O₂— and O₂-treated rabbits (p=0.0665). Also, there was no significant difference in the TTC probability between the O₂ and sham groups (p=0.5781). Statistically significant changes were marked with p<0.05*; p<0.001***. ns=not significant. Shown in brackets are the numbers and percentages of surviving animals on day 90.

FIG. 3

Growth and development of the VX2 tumor cells in the right ear of NZW rabbits after inoculation. Panel (a) shows representative macroscopic views of a solid auricular VX2 tumor in the right ear of a rabbit on day 14 after tumor cell inoculation and different stages of remission after O₃/O₂ therapy (O₃/O₂-pneumoperitoneum). The spontaneous tumor remission observed in one sham-treated rabbit and the two remissions after O₂ gas insufflation were similar (macroscopic views are not shown). Note, that only a small scar of the remitted auricular tumor remained on day 90, the end point of our observation period. In sharp contrast, in rabbits that succumbed to tumor progression the auricular tumor continued growing resulting in severe ulcerations associated with massive bleeding and onset of local infections introducing the final stage of this tumor disease (b, representative stages of the same rabbit are shown). Depicted are tumor stages on day 14, 27, 35, and at the end of the observation period (90 days or 42 days in case of death).

FIG. 4

VX2 tumor derived distant lung metastases.

CT scans of the thorax showing no detectable lung metastases in a rabbit of the O₃/O₂-rem group (a) at the end of the observation period, but reveals a huge metastasis in the lung of a rabbit on day 32 (b, asterisk) that succumbed to the VX2 tumor later on. (depicted are representative CT scans of the lungs amounting to the vena pulmonalis) The lower images show macroscopic views of the complete lungs of a healthy rabbit (c) and of an animal with multiple VX2 carcinoma derived lung metastases at the pleura visceralis (d).

FIG. 5

Course of mean body weight from day zero (at which VX2 cell suspension was inoculated) until day forty-two. On day zero the initial body weight of each rabbit was set to 100 percent; changes were expressed in percentage of initial body weight. The period of the therapeutic O₃/O₂ gas mixture insufflation is marked by a gray box. Changes in the body weight within an experimental group were calculated for each day by the Student's t test. Significant loss of body weight was found in the O₃/O₂-treated group (*p<0.05; **p<0.01) and in the O₂-treated group (^{#}p<0.05) compared to the mean body weight of each group on day 14 before initiation of treatment. The body weight change in the sham group was not significant.

1. Use of a gaseous medical ozone/oxygen mixture for the production of a medicament for the primary therapy of highly metastasizing tumors.
